(19) United States
(12) Patent Application Publication
(10) Pub. No.: US 2017/0000119 A1
(43) Pub. Date: Jan. 5, 2017

(54) Microbicidal Polymers And Methods Of Use Thereof

(71) Applicant: XMICROBIAL, LLC, Scituate, MA (US)

(72) Inventors: Roger A. Nassar, Swampscott, MA (US); Shirley Young, Scituate, MA (US); Marion L. Chiattello, Cedar Falls, IA (US); Mark Oman, West Des Moines, IA (US)

(21) Appl. No.: 15/106,597

(22) PCT Filed: Dec. 22, 2014
(86) PCT No.: PCT/US14/71823

§ 371 Date: Jun. 20, 2016

Related U.S. Application Data
(60) Provisional application No. 61/920,299, filed on Dec. 23, 2013.

(51) Int. Cl. A01N 37/20; A01N 33/12; A61L 2/18; C08G 65/00; C09D 171/02; A61L 2/232; A01N 25/08; C09D 5/14

(52) U.S. Cl. CPC A01N 37/20; A01N 25/08; A01N 33/12; C09D 5/14; C08G 65/00; C09D 171/02; A61L 2/232; A61L 2/18; C08G 2150/00

(57) ABSTRACT

Provided herewith are microbicidal polymers comprising an alkylene oxide backbone comprising one or more alkyl and/or alkylene oxide primary branches, in which at least one of the alkyl and/or alkylene oxide primary branches is functionalized with a quaternary ammonium or fluorinated group, or at least two of the alkyl and/or alkylene oxide primary branches are functionalized with a quaternary ammonium and fluorinated group, the primary branches optionally contain one or more alkyl and/or alkylene oxide secondary branches that are functionalized with a quaternary ammonium or fluorinated group, compositions comprising the polymer and a carrier, and methods of using the same.

### FIELD

The present disclosure is directed, in part, to microbicidal polymers comprising an alkylene oxide backbone and methods of using the same.

### BACKGROUND

Pathogenic microbes such as bacteria, virus, fungi, yeast, and algae, are known to cause serious illnesses and death. Microbes are found on common objects such as doorknobs, elevator buttons, faucets, kitchen counters, and farming equipment that people come into contact in their daily lives. People and animals that come into contact with such microbes are at risk or infected by these microbes. There is therefore a need to kill or inactivate the microbes or prevent the surfaces from being invaded by the microbes.

Polymeric coatings have been proposed for inactivating microbes such as bacteria and virus. For example, United States Patent Application Publication No. 2010/0136072 A1 discloses hydrophobic, water-insoluble, charged polymers based on polyethyleneimine (PEI). Larson et al. (Biotech. and Bioeng., 108, 720-723 (2011)) discloses certain hydrophobic polycationic coatings for disinfecting poliovirus and rotavirus. Hsu et al. (Biotechnol. Lett., 33, 411-416 (2011)) discloses that certain N-alkylated polyethyleneimine polymers kill E. coli and Staphylococcus aureus on contact. However, some of these polymers are soluble only in harsh organic solvents such as tert-butanol. Thus, application of these polymers for microbicidal use on surfaces to be disinfected is not safe to the person who applies the coatings; such solvents are also unsafe to the environment.

The foregoing shows that there exists an unmet need for polymers and microbicidal compositions which are soluble in less harsh solvents such as water or lower alcohols and which are safe to the person applying the coating and to the environment.

### SUMMARY

One or more of the foregoing needs have been fulfilled by the present disclosure. Accordingly, the disclosure provides a polymer comprising an alkylene oxide backbone to which are attached one or more alkyl or alkylene oxide or a combination of alkyl and alkyene oxide primary branches, wherein at least one of the primary branches is functionalized with a quaternary ammonium group or a fluorinated group, or at least two of the primary branches are functionalized with a quaternary ammonium group and a fluorinated group, wherein the primary branches optionally contain one or more alkyl or alkylene oxide secondary branches that are functionalized with a quaternary ammonium group or a fluorinated group, wherein said polymer is associated with an anion to maintain electro-neutrality when a quaternary ammonium group is present.

The disclosure further provides a composition comprising (i) a polymer as described herein; and (ii) a carrier.

Also provided are methods of using a polymer as described herein, particularly a composition thereof. The inventive methods include a method of coating a surface, a method of disinfecting a surface, and a method of protecting a surface against growth of a microorganism.

Because of the presence of two main groups having different chemical characteristics, water-soluble backbone (alkylene oxide) and organic solvent-soluble functionalities (ammonium and/or fluorinated groups), the solubility of the microbicidal polymer can be tuned to be high in non-hazardous solvents, such as water or low molecular weight alcohols. Furthermore, it can be tuned to be high either in water or in organic solvent, or in a specific combination of solvents composed of water and one or more organic solvents, or in a combination of two or more organic solvents.

Additionally, because of the presence of two main groups having different chemical characteristics, namely, a hydrophilic backbone (alkylene oxide) and hydrophobic and/or fluorophilic functionalities (ammonium and/or fluorinated groups), the overall performance and interaction of the microbicidal polymer with its surrounding chemicals and surfaces can be tuned. For example, shorter alkylene in alkylene oxide groups and/or higher repeating number of alkylene oxide groups and/or shorter alkyl groups in ammoniums and/or shorter fluorinated groups lead to a more hydrophilic character in the microbicidal polymer. Such character generates stronger interactions between the polymer and surrounding hydrophilic entities. Conversely, longer alkylene in alkylene oxide groups and/or lower repeating number of alkylene oxide groups and/or longer alkyl groups in ammoniums and/or longer fluorinated groups all lead to a more hydrophobic character in the microbicidal polymer. Such character generates weaker interactions between the polymer and surrounding hydrophilic entities.

Moreover, coatings of the microbicidal polymers can be removed from a surface, thereby regenerating the surface as needed.

### BRIEF DESCRIPTION OF THE DRAWINGS

FIG. 1 depicts a two-step process for functionalizing 8-arm PEG with alkyl ammonium groups to form polycation N,N-decyl,methyl-PEG(10 k, 8-arm) in accordance with an embodiment of the disclosure.

FIG. 2 depicts a one-step process for functionalizing 8-arm PEG with nonafluorobutyl groups to form N-nonafluorobutylamide-PEG(10 k, 8-arm) in accordance with an embodiment of the disclosure.

FIG. 3 depicts a three-step process for functionalizing 8-arm PEG with alkyl ammonium and nonafluorobutyl groups in an equal ratio to form polycation (N,N-dodecyl,methyl)₄(N-nonafluorobutylamide)₄-PEG(10 k, 8-arm)) in accordance with an embodiment of the disclosure.

FIG. 4 is a series of photographs that depict (a) an untreated glass slide, (b) a glass slide pretreated with 70% isopropanol, and (c) a glass slide pretreated with polycation N,N-decyl,methyl-PEG(10 k, 8-arm).

FIG. 5 is a series of photographs that depict (a) an untreated glass slide, (b) a glass slide pretreated with 70% isopropanol, and (c) a glass slide pretreated with polycation N,N-decyl,methyl-PEG(10 k, 8-arm) in 70% isopropanol.

### DESCRIPTION OF EMBODIMENTS

The present disclosure provides polymers comprising an alkylene oxide backbone to which are attached one or more alkyl or alkylene oxide or a combination of alkyl and alkylene oxide primary branches, wherein at least one of the primary branches is functionalized with a quaternary ammonium group or a fluorinated group, or at least two of the primary branches are functionalized with a quaternary ammonium group and a fluorinated group, wherein the primary branches optionally contain one or more alkyl or alkylene oxide secondary branches that are functionalized with a quaternary ammonium group or a fluorinated group, wherein said polymer is associated with an anion to maintain electro-neutrality when a quaternary ammonium group is present.

The anion is any suitable negatively charged moiety that serves to neutralize the charge of a quaternary ammonium group, as described herein. The anion can be, for example, a halide (e.g., Cl⁻, F⁻, Br⁻, I⁻), an oxoanion (e.g., CO₃²⁻, HCO₃²⁻, OH⁻, NO³⁻, PO₄³⁻, or SO₄²⁻), or an organic anion (e.g., CH₃COO⁻, HCOO⁻, C₂O₄²⁻, or CN⁻).

The alkylene oxide backbone, alkyl or alkylene oxide primary branches, and alkyl or alkylene oxide secondary branches can have any number of suitable carbons, such as C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀. The number of carbons in the alkylene oxide backbone or alkyl or alkylene oxide primary or secondary branches is determined by the desired solubility properties and/or end use. For example, it will generally be understood that the larger the alkylene portion of the alkylene oxide backbone, primary branches, and/or secondary branches, and the longer the alkyl group in the primary branches and/or secondary branches, the less water or lower alcohol soluble the polymer will be. Inversely, the shorter the alkylene portion of the alkylene oxide backbone, primary branches, and/or secondary branches, and the shorter the alkyl group in the primary branches and/or secondary branches, the more water or lower alcohol soluble the polymer will be.

In some aspects, the alkylene oxide backbone comprises a propylene (C₃) oxide backbone, an ethylene (C₂) oxide backbone, or both propylene oxide and ethylene oxide units. In some embodiments, the primary branch and/or secondary branch comprises at least one ethylene oxide unit or at least one propylene oxide unit.

In certain aspects, the alkylene oxide backbone is a propylene oxide of the formula:

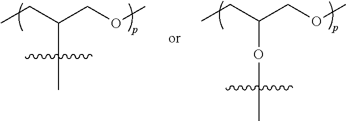

wherein p is 1 to 60. For example, p is a range of 1 to 50, 1 to 40, 1 to 30, 1 20, 2 to 20, 2 to 15, 2 to 12, 2 to 10, 2 to 8, 3 to 10, 3 to 8, 4 to 10, 4 to 8, 5 to 10, 5 to 8, 6 to 10, or 6 to 8; or p is 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 52, 54, 56, 58, or 60. The alkyl and/or alkyene oxide primary branches are linked to one or more of the three open sites shown above.

The alkylene oxide backbone can be linked to any suitable number of alkyl and/or alkylene oxide primary or secondary branches. In some embodiments, the length of the alkyl branches and the number of primary or secondary branches is determined by the desired solubility properties and/or end use. In certain aspects, the alkylene oxide backbone comprises at least 2 (e.g., at least 3, at least 4, at least 4, at least 5, at least 6, at least 7, or at least 8) primary or secondary branches. The backbone can have an upper limit of any number of suitable branches, e.g., up to 100 (e.g., up to 80, up to 60, up to 40, up to 20, or up to 10) alkyl and/or alkylene oxide primary or secondary branches. These lower and upper limits with respect to the number of alkyl and/or alkylene oxide primary or secondary branches can be used in any combination (e.g., 2-100, 3-80, and 4-10, etc.). In certain aspects, the backbone has 2 to 20 primary branches. In some embodiments, the alkylene oxide backbone is linked to 4 to 8 primary branches (e.g., ethylene oxide primary branches). Depending on the number and placement of branches, the polymer can be described as a star polymer, comb polymer, brush polymer, palm tree polymer, H-shaped polymer, or dumbbell polymer.

In some embodiments, the primary or secondary branch can be based on polyethylene glycol (PEG). For example, a branched PEG, e.g., an alkylene oxide backbone comprising 2 to 100 PEG branches, can be used as the microbicidal polymer in accordance with an embodiment. In some embodiments, the polymer comprises 3 to 10 (e.g., 3, 4, 5, 6, 7, 8, 9, or 10) PEG branches.

In some embodiments, the primary branch or the secondary branch, that is functionalized is of the formula

—(CH₂)ₙ—X,—(CH₂CH₂O)ₙ—X, or —(CH₂CH₂CH₂O)ₙ—X,

wherein:

X is —(CH₂)ₘ—Y, when Y is a quaternary ammonium group as described herein or

X is —(CH₂)ₘ—NHC(O)—Y, when Y is a fluorinated group as described herein,

m is 0 to 10, and

n is 1 to 2,500.

The value of m determines the length of the alkylene linker (e.g., 0, 1, 2, 3, 4, 5, 6, 7, 8, 9, or 10). If no linker is necessary, then m is 0. In certain embodiments, m is 1 or 2.

The value of n determines, in part, the molecular weight of the polymer. The molecular weight of the polymer is as described herein, and n is a number that provides the desired molecular weight. Typically n is 1 to 2,500 (e.g., 1 to 2,000, 2 to 1,000, 2 to 800, 2 to 600, 3 to 500, 3 to 400, 4 to 400, 4 to 300, etc.).

The microbicidal polymer of the disclosure can be provided or prepared by any suitable method. For example, synthesis methods are described herein, and polymer starting materials can be prepared by methods known in the art or are commercially available (e.g., Sigma-Aldrich (St. Louis, Mo.), Dow Chemical (Midland, Mich.), JenKem Technology (Allen, Tex.)). Typical polymerization methods include ring opening polymerization, suspension polymerization, free radical polymerization, anionic polymerization, cationic polymerization, or metallocene catalysis and can include an initiator and/or a catalyst. Examples of a suitable catalyst include an acid catalyst, an alkali metal-based catalyst (e.g., NaOH, KOH, Na₂CO₃), a metal oxide catalyst, an Mg-based catalyst, a Ca-based catalyst, an Al-based catalyst, and a combination thereof.

The microbicidal polymer can be any suitable average molecular weight and usually is a function of the ratio of starting materials and synthesis method. Typically, the molecular weight is tuned based on the desired solubility properties and/or end use. For example, the number, weight, or volume average molecular weight can be at least about 200 g/mol (e.g., at least about 300 g/mol, at least about 500 g/mol, at least about 800 g/mol, at least about 1,000 g/mol, at least about 1,500 g/mol, at least about 2,000 g/mol) and/or up to about 100,000 g/mol (e.g., up to about 90,000 g/mol, up to about 80,000 g/mol, up to about 70,000 g/mol, up to about 60,000 g/mol, up to about 50,000 g/mol, up to about 40,000 g/mol, up to about 30,000 g/mol, up to about 20,000 g/mol, or up to about 10,000 g/mol). These lower and upper limits with respect to the number, weight, or volume average molecular weight can be used in any combination to describe the polymer molecular weight range (e.g., about 200 to about 100,000 g/mol, about 300 g/mol to about 50,000 g/mol, and about 1,000 to about 20,000 g/mol, etc.).

The polymer can be characterized quantitatively using known methods. For example, molecular weight determinations can be made using gel permeation chromatography (also known as size exclusion chromatography and gel filtration chromatography), nuclear magnetic resonance spectroscopy (NMR), matrix-assisted laser desorption/ionization mass spectroscopy (MALDI), light scattering (e.g., low angle and multi angle), small angle neutron scattering (SANS), sedimentation velocity, end group analysis, osmometry, cryoscopy/ebulliometry, and viscometry.

In some aspects, at least one of the alkyl and/or alkylene oxide primary branches is functionalized with a quaternary ammonium group (such as substituent Y described above), which can have the formula —N⁺R¹R²R³. Substituents R¹, R², and R³ are independently selected from alkyl, alkenyl, cycloalkyl, and aryl. In some embodiments, R¹, R², and R³ are selected based on the desired solubility properties and/or end use. For example, it will generally be understood that the larger the number of carbons in R¹, R², and/or R³, the less water and/or lower alcohol soluble the polymer will be. Inversely, the fewer the number of carbons in R¹, R², and/or R³, the more water and/or lower alcohol soluble the polymer will be. In some embodiments, R¹, R², and R³ are independently alkyl, such as a C₁₋₂₀ alkyl (e.g., C₁₋₁₈ alkyl, C₁₋₆ alkyl, C₁₋₁₄ alkyl, C₁₋₁₂ alkyl, or C₁₋₁₀ alkyl). In specific examples, R¹ and R² are each a lower alkyl (e.g., methyl, ethyl, propyl, butyl, or pentyl) and R³ is an alkyl selected from hexyl, octyl, decyl, dodecyl, tetradecyl, hexadecyl, and octadecyl. In some embodiments, R¹ and R² are methyl, and R³ is decyl, dodecyl, or octadecyl.

In some aspects, at least one of the alkyl and/or alkylene oxide primary branches is functionalized with a fluorinated group (such as substituent Y described above). The fluorinated group can be, for example, fluoroalkyl, fluoroalkenyl, fluorocycloalkyl, or fluoroaryl). In some embodiments, the fluorinated group is perfluoroalkyl, such as C₁₋₈ perfluoroalkyl (e.g., C₁₋₁₆ perfluoroalkyl, C₁₋₁₄ perfluoroalkyl, C₁₋₁₂ perfluoroalkyl, or C₁₋₁₀ perfluoroalkyl). In some embodiments, the fluoroalkyl is nonafluorobutyl and its isomers, heptafluoropropyl and its isomers, and pentafluoroethyl.

In certain other aspects, the polymer comprises at least two primary branches. One of primary branches is functionalized with a quaternary ammonium group, and the other primary branch is functionalized with a fluorinated group. This type of hybrid polymer can have any number of quaternary ammonium or fluorinated groups (e.g., at least 1 of each, at least 2 of each, at least 3 of each, at least 4 of each, at least 5 of each, at least 10 of each, at least 15 of each, at least 20 of each, etc.). The polymer can comprise equal or unequal numbers of quaternary ammonium and fluorinated groups (e.g., 3 fluorinated groups and 1 quaternary ammonium group; or 4 fluorinated groups and 4 quaternary ammonium groups). The quaternary ammonium group and fluorinated group are as described herein.

Provided is a method of adjusting the solvation of the polymer compositions described herein comprising selecting an appropriate alkylene oxide and alkyl groups in the backbone and branches and their number of repeat units and density of branches, and selecting a quaternary ammonium group or groups and/or a fluorinated group or groups to provide a polymer that is soluble in water, organic solvent, or a mixture thereof. The solvation includes, for example, dissolution, hydration, and/or solvent association of a polymer composition, which can occur to any degree.

Further provided is a method of adjusting the hydrophobicity, hydrophilicity, and/or fluorophilicity of the polymer compositions described herein comprising selecting an appropriate alkylene oxide and alkyl groups in the backbone and branches and their number of repeat units and density of branches, and selecting a quaternary ammonium group or groups and/or a fluorinated group or groups to provide a polymer that is hydrophobic, hydrophilic, and/or fluorophilic.

As used herein, unless otherwise specified, the term “alkyl” means a saturated straight chain or branched non-cyclic hydrocarbon having an indicated number of carbon atoms (e.g., C₁-C₂₀, C₁-C₁₈, C₁-C₁₆, C₁-C₁₄, C₁-C₁₂, C₁-C₁₀, C₁-C₈, C₁-C₆, C₁-C₄, etc.). Representative saturated straight chain alkyls include methyl, ethyl, n-propyl, n-butyl, n-pentyl, n-hexyl, n-heptyl, n-octyl, n-nonyl, n-decyl, n-dodecyl, n-tetradecyl, n-hexadecyl, and n-octadecyl; while representative saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. An alkyl group can be unsubstituted or substituted.

As used herein, unless otherwise specified, the term “alkenyl group” means a straight chain or branched non-cyclic hydrocarbon having an indicated number of carbon atoms (e.g., C₂-C₂₀, C₂-C₁₈, C₂-C₁₆, C₂-C₁₄, C₂-C₁₂, C₂-C₁₀, etc.) and including at least one carbon-carbon double bond. Representative straight chain and branched alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, and the like. Any unsaturated group (double bond) of an alkenyl can be unconjugated or conjugated to another unsaturated group. An alkenyl group can be unsubstituted or substituted.

The term “cycloalkyl,” as used herein, means a cyclic alkyl moiety containing from, for example, 3 to 7 carbon atoms, or from 5 to 6 carbon atoms. Examples of such moieties include cyclopropyl, cyclobutyl, cyclopentyl, cyclohexyl, and the like.

The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2π electrons, according to Hückel's Rule, wherein n=1, 2, or 3.

As used herein, unless otherwise specified, the term “substituted” means a group substituted by one or more substituents (e.g., 1, 2, 3, 4, 5, 6, etc.), such as, alkyl, alkenyl, alkynyl, cycloalkyl, aroyl, halo, haloalkyl (including trifluoromethyl), haloalkoxy (including trifluoromethoxy), hydroxy, alkoxy, cycloalkyloxy, heterocyclooxy, oxo (═O), alkanoyl, aryl, arylalkyl, alkylaryl, heteroaryl, heteroarylalkyl, alkylheteroaryl, heterocyclo, aryloxy, alkanoyloxy, amino, alkylamino, arylamino, arylalkylamino, cycloalkylamino, heterocycloamino, alkanoylamino, aroylamino, aralkanoylamino, substituted alkanoylamino, substituted arylamino, substituted aralkanoylamino, thiol (mercapto), alkylthio, arylthio, arylalkylthio, cycloalkylthio, heterocyclothio, alkylthiono, arylthiono, arylalkylthiono, alkylsulfonyl, arylsulfonyl, arylalkylsulfonyl, sulfonamido (e.g., —SO₂NH₂), substituted sulfonamido, nitro, cyano, carboxy, carbamido, carbamyl (e.g., —CONH₂), substituted carbamyl (e.g., —CONH-alkyl, —CONH-aryl, —CONH-arylalkyl, or instances where there are two substituents on the nitrogen selected from alkyl or arylalkyl), alkoxycarbonyl, aryl, substituted aryl, substituted or unsubstituted heterocycloalkyl, and substituted or unsubstituted heteroaryl.

In some embodiments, the polymer is selected from

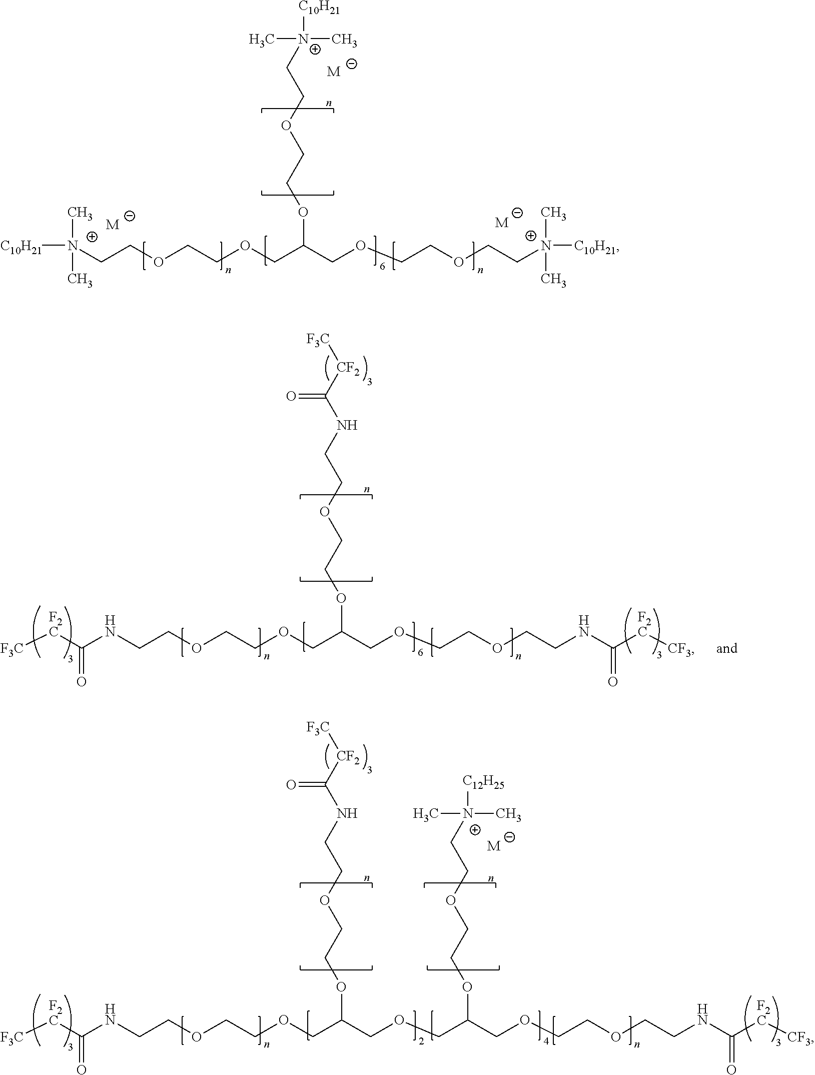

wherein M⁻ is an anion, and n is 2 to 2500.

The anion M⁻ is any suitable negatively charged moiety that serves to neutralize the charge of a quaternary ammonium group, as described herein. The anion can be, for example, a halide (e.g., Cl⁻, F⁻, Br⁻, I⁻), an oxoanion (e.g., CO₃²⁻, HCO₃²⁻, OH⁻, NO^{3-,} PO₄³⁻, or SO₄²⁻), Or an organic anion (e.g., CH₃COO⁻, HCOO⁻, C₂O₄²⁻, or CN⁻). The substituent n is as described herein and determines, at least in part, the molecular weight of the polymer.

Other examples of the microbicidal polymer include: Polycation N,N-decyl,methyl-PEG(10 k, 8-arm); Polycation N,N-decyl,methyl-PEG(10 k, 4-arm); Polycation N,N-decyl,methyl-PEG(20 k, 8-arm); Polycation N,N-decyl,methyl-PEG(20 k, 4-arm); Polycation N,N-decyl,methyl-PEG(40 k, 8-arm); Polycation N,N-decyl,methyl-PEG(40 k, 4-arm); Polycation N,N-octyl,methyl-PEG(10 k, 8-arm); Polycation N,N-octyl,methyl-PEG(10 k, 4-arm); Polycation N,N-octyl,methyl-PEG(20 k, 8-arm); Polycation N,N-octyl,methyl-PEG(20 k, 4-arm); Polycation N,N-octyl,methyl-PEG(40 k, 8-arm); Polycation N,N-octyl,methyl-PEG(40 k, 4-arm); Polycation N,N-hexyl,methyl-PEG(10 k, 8-arm); Polycation N,N-hexyl,methyl-PEG(10 k, 4-arm); Polycation N,N-hexyl,methyl-PEG(20 k, 8-arm); Polycation N,N-hexyl,methyl-PEG(20 k, 4-arm); Polycation N,N-hexyl,methyl-PEG(40 k, 8-arm); Polycation N,N-hexyl,methyl-PEG(40 k, 4-arm); N-nonafluorobutylamide-PEG(10 k, 8-arm); N-nonafluorobutylamide-PEG(10 k, 4-arm); N-nonafluorobutylamide-PEG(20 k, 8-arm); N-nonafluorobutylamide-PEG(20 k, 4-arm); N-nonafluorobutylamide-PEG(40 k, 8-arm); N-nonafluorobutylamide-PEG(40 k, 4-arm); Polycation (N,N-dodecyl,methyl)₄(N-nonafluorobutylamide)₄-PEG(10 k, 8-arm); Polycation (N,N-dodecyl,methyl)₂(N-nonafluorobutylamide)₆-PEG(10 k, 8-arm); Polycation (N,N-dodecyl,methyl)₆(N-nonafluorobutylamide)₂-PEG(10 k, 8-arm); Polycation (N,N-dodecyl,methyl)₄(N-nonafluorobutylamide)₄-PEG(20 k, 8-arm); Polycation (N,N-dodecyl,methyl)₂(N-nonafluorobutylamide)₆-PEG(20 k, 8-arm); Polycation (N,N-dodecyl,methyl)₆(N-nonafluorobutylamide)₂-PEG(20 k, 8-arm); Polycation (N,N-dodecyl,methyl)₄(N-nonafluorobutylamide)₄-PEG(40 k, 8-arm); Polycation (N,N-dodecyl,methyl)₂(N-nonafluorobutylamide)₆-PEG(40 k, 8-arm); Polycation (N,N-dodecyl,methyl)₆(N-nonafluorobutylamide)₂-PEG(40 k, 8-arm); Polycation (N,N-hexyl,methyl)₄(N-nonafluorobutylamide)₄-PEG(10 k, 8-arm); Polycation (N,N-hexyl,methyl)₂(N-nonafluorobutylamide)₆-PEG(10 k, 8-arm); Polycation (N,N-hexyl,methyl)₆(N-nonafluorobutylamide)₂-PEG(10 k, 8-arm); Polycation (N,N-hexyl,methyl)₄(N-nonafluorobutylamide)₄-PEG(20 k, 8-arm); Polycation (N,N-hexyl,methyl)₂(N-nonafluorobutylamide)₆-PEG(20 k, 8-arm); Polycation (N,N-hexyl,methyl)₆(N-nonafluorobutylamide)₂-PEG(20 k, 8-arm); Polycation (N,N-hexyl,methyl)₄(N-nonafluorobutylamide)₄-PEG(40 k, 8-arm); Polycation (N,N-hexyl,methyl)₂(N-nonafluorobutylamide)₆-PEG(40 k, 8-arm); and Polycation (N,N-hexyl,methyl)₆(N-nonafluorobutylamide)₂-PEG(40 k, 8-arm).

The present disclosure provides a composition comprising:

(i) at least one of any of the polymers described herein; and

(ii) a carrier.

Any suitable carrier known in the art can be used. The choice of the carrier will be determined, in part, both by the particular composition (e.g., the chemical structure of the polymer and its physical properties) and by the particular method of using the composition. Accordingly, there are a wide variety of suitable formulations of the composition of the present disclosure. Suitable formulations include a solid, a liquid (e.g., a solution, a suspension, or a dispersion), and a gel.

In an embodiment, a solvent is used as the carrier. Typical solvents include for example, water, saline, an alcohol, propylene glycol, acetonitrile, low molecular weight polyethylene glycol, glycerol, and acetic acid. In some embodiments, water and lower alcohols such as methanol, ethanol, or isopropanol are used. In some embodiments, the solvent does not include the use of solvents typically considered harsh or hazardous, such as inorganic solvents other than water or certain organic solvents that are considered carcinogenic, a reproductive hazard, and/or a neurotoxin. Hazardous solvents are known in the art and are classified by, e.g., the National Institute for Occupational Safety and Health (NIOSH). Classes of hazardous organic solvents include aliphatic hydrocarbons, aromatic hydrocarbons, nitrated hydrocarbons, chlorinated hydrocarbons, amines, esters, ethers, and ketones. Organic solvents that are not included in the composition include n-butanol, t-butanol, benzene, toluene, turpentine, dichloromethane, tetrachloroethylene, chloroform, methyl acetate, ethyl acetate, acetone, hexane, cyclohexane, petrol ether, and dimethylformamide.

In some aspects, the carrier is a solvent that is an organic solvent, water, or a mixture thereof, wherein the polymer is dissolved in the solvent. In some embodiments, the solvent comprises an alcohol, such as a lower alcohol (e.g., methanol, ethanol, n-propanol, isopropanol, or a combination thereof). In some embodiments, the alcohol is methanol, ethanol, isopropanol, or a combination thereof. The mixture of water and/or alcohols can be in any suitable proportion. For example, when two solvents (e.g., water and ethanol; ethanol and isopropanol) are used, the ratios can range from 1/99 to 99/1 (e.g., 5/95, 10/90, 15/85, 20/80, 25/75, 30/70, 35/65, 40/60, 45/55, or 50/50).

When the composition is a liquid comprising a solvent, the formulation can be provided in any suitable manner for a desired end use (e.g., as a mist, spray, a wipe or cloth that has been soaked in the liquid, or a wash bottle liquid).

Advantageously, when the solvent comprises alcohol, the resulting composition has a dual action. As a first action, the alcohol kills microbes immediately after application of the composition onto a surface. As a second, prolonged action, the polymer generates a long-lasting microbicidal coating on the surface. For example, the coating containing quaternary ammonium groups act to kill or inactivate the microbes that come into contact with the coating; additionally, or alternatively, when the coating contains a fluorinated group, microbes are prevented from adhering to the surface.

In accordance with embodiments of the disclosure, the composition can be a solid formulation such as, but not limited to, a foam, film, woven or non-woven material, hydrogel, ointment, salve, cream, gel matrix, and mixtures and blends thereof. Solid formulations can include, for example, povidone, copovidone (a copolymer of vinyl acetate and polyvinylpyrrolidone), crospovidone, lactose, mannitol, cornstarch, potato starch, microcrystalline cellulose, acacia, gelatin, colloidal silicon dioxide, croscarmellose sodium, talc, magnesium stearate, stearic acid, and other excipients. In certain aspects, the composition is a personal care formulation, a cosmetic formulation, animal care formulation, or a detergent formulation.

Any of the compositions described herein can comprise additional components, as determined, in part, by the polymer and/or the desired end use. Additional components include, e.g., colorants, diluents, buffering agents, moistening agents, suspending agents, solubilizers, thickening agents, stabilizers, preservatives, and an additional active agent (e.g., another antibacterial agent, antifungal agent, or antiviral agent).

The microbicidal polymer of the disclosure can be present in the composition in any suitable concentration. Typically, the concentration will be an amount effective to provide a desired result, e.g., provide a microbicidal effect. In some embodiments, the amount of polymer is an amount sufficient such that the polymer is soluble in a particular carrier (e.g., solvent). Examples of concentrations of polymer include at least 0.001 part by weight polymer relative to 1 part by volume of carrier (e.g., at least 0.002 part by weight polymer, at least 0.005 part by weight polymer, at least 0.01 part by weight polymer, at least 0.015 part by weight polymer, at least 0.02 part by weight polymer, at least 0.025 part by weight polymer, at least 0.03 part by weight polymer, at least 0.04 part by weight polymer, at least 0.05 part by weight polymer, at least 0.06 part by weight polymer, at least 0.08 part by weight polymer, at least 0.1 part by weight polymer, at least 0.2 part by weight polymer, at least 0.3 part by weight polymer, at least 0.4 part by weight polymer, at least 0.5 part by weight polymer, at least 0.6 part by weight polymer, at least 0.8 part by weight polymer, and at least 1 part by weight polymer). The maximum amount of polymer is not particularly limited, but typically will be about 10 parts by weight polymer relative to 1 part by volume of carrier or less (e.g., about 9 parts by weight polymer or less, about 8 parts by weight polymer or less, about 7 parts by weight polymer or less, about 6 parts by weight polymer or less, about 5 parts by weight polymer or less, about 4 parts by weight polymer or less, about 3 parts by weight polymer or less, about 2 parts by weight polymer or less, about 1 parts by weight polymer or less, or about 0.5 parts by weight polymer or less).

The disclosure further provides methods of using a microbicidal polymer as described herein. Typically, the polymer will be used in the form of a composition. Settings suitable for the use of the polymer described herein include, but not limited to, a home, office, hospital, clean room, research lab, veterinary settings, factory, animal processing plants, construction site, public facility, dormitory, school, airport, stadium, park, playground, vehicles for air, land, and water, and agricultural environments.

In particular, the disclosure provides a method of coating a surface comprising applying to the surface a composition as described herein, and evaporating the solvent to form a coating on the surface. The coating can fully or partially cover the surface. With such a coating, it is believed that microbes that land on the surface are either repelled or rendered inactive. The composition can be applied by any suitable method, such as spraying, misting, rolling, brushing, dipping, dip-coating, laminating, extrusion, vapor deposition, knife coating, gravure coating, hot melt coating, silk screen coating, slot die coating, spin coating, and lithography. The composition can also be introduced into the production process of the surface.

In accordance with this embodiment, the disclosure provides a coated surface comprising a surface and a polymer composition as described herein that is coated on the surface. Provided is a method of adjusting the hydrophobicity, hydrophilicity, fluorophilicity, and/or microbicidal performance of the coated surface described herein. The method comprises selecting an appropriate concentration of polymer on the surface by varying the polymer concentration in the carrier and by varying the number and methods of polymer coating.

Also provided is a method of disinfecting a surface comprising applying to the surface a composition as described herein. The method of disinfecting can include, e.g., repelling, destroying, killing, and/or deactivating a microbe or microorganism.

Further provided is a method of protecting a surface against growth of a microorganism comprising applying to the surface a composition as described herein. The method of protecting a surface against growth of a microorganism can include, e.g., repelling, destroying, killing, and/or deactivating a microorganism. Because of the application of the microbicidal polymer, the growth of the microorganism on a surface is reduced compared to the amount of microorganism growth on the same surface, under the same conditions (e.g., temperature, relative humidity, light level, etc.), without the presence of the microbicidal polymer. The level of reduction of growth can be any level, including a 100% (e.g., 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, or 10%) reduction in growth.

A “microorganism” (i.e., a microbe) as used herein can be a single cell or multicellular organism and includes organisms such as prokaryotes (e.g., bacteria and archaea), eukaryotes (e.g., protozoa, fungi, algae, microscopic plants and animals), and viruses. For example, the bacteria can be gram negative or gram positive. In specific embodiments, the microorganism is selected from Staphylococcus aureus, Streptococcus, Escherichia coli (E. coli), Pseudomonas aeruginosa, mycobacterium, adenovirus, rhinovirus, smallpox virus, influenza virus, herpes virus, human immunodeficiency virus (HIV), rabies, chikungunya, severe acute respiratory syndrome (SARS), malaria, dengue fever, tuberculosis, meningitis, typhoid fever, yellow fever, ebola, shingella, listeria, yersinia, West Nile virus, protozoa, fungi Salmonella enterica, Candida albicans, Trichophyton mentagrophytes, poliovirus, Enterobacter aerogenes, Salmonella typhi, Klebsiella pneumonia, Aspergillus brasiliensis, and methicillin resistant Staphylococcus aureus (MRSA).

Without being bound by any particular theory, it is believed that the microbicidal polymers described herein are effective by deactivating microbes, repelling microbes, or a combination of both deactivating and repelling microbes. For example, a polymer comprising one or more quaternary ammonium groups (e.g., a “polycation” polymer) can be coated onto a surface. A microbe can come into contact with the coated surface and then be killed (e.g., cell membrane can rupture) due to the physical structure of the cation. It is believed that long hydrophobic alkyl substituents R¹, R², and/or R³ in the functionality —N⁺R¹R²R³ penetrate the membranes of the microbes with which it comes in contact. The penetration of the alkyl substituent(s) through the membrane of the microbe presumably leads to the rupturing of the membrane and killing of the microbe. This phenomenon has been compared to a “bubble-bursting porcupine” model. The killing or rupturing leads to the inactivation of the microbe.

In another example, a polymer comprising one or more fluorinated groups can be coated onto a surface. It is believed that the fluorinated groups provide a non-reactive and inert character to the surface, hindering the attachment of microbes and their nutrients to the surface. The inert character of highly fluorinated surfaces is well known in the literature as well as in commercial products (e.g., TEFLON™, fluorinated plastics in implants, and graft materials in surgical interventions). A microbe can diffuse onto or approach the surface but will be repelled by the physical structure of the fluorinated group. If the microbe adheres to the surface, its life is shortened due to an absence of nutrients. A polymer comprising at least one quaternary ammonium group and at least one fluorinated group can act via a combination of both mechanisms.

The surface that is being disinfected or protected can be of any suitable material, including a biocompatible material. The surface can be used in or derived from any suitable form, such as, for example, a powder, dust, an aggregate, an amorphous solid, a sheet, a fiber, a tube, a fabric, or the like. In embodiments, the surface comprises metal, glass, fiberglass, silica, sand, wood, fiber, natural polymer, synthetic polymer, plastic, rubber, ceramic, porcelain, stone, marble, cement, or human or animal body.

Metal surfaces suitable for use in the disclosure include, for example, stainless steel, nickel, titanium, tantalum, aluminum, copper, gold, silver, platinum, zinc, Nitinol, Inconel, iridium, tungsten, silicon, magnesium, tin, alloys, coatings containing any of the foregoing, galvanized steel, hot dipped galvanized steel, electrogalvanized steel, annealed hot dipped galvanized steel, and combinations thereof.

Glass surfaces suitable for use in the disclosure include, for example, soda lime glass, strontium glass, borosilicate glass, barium glass, glass-ceramics containing lanthanum as well as combinations thereof.

Silica surfaces suitable for use in the disclosure include, for example, quartz, fused quartz, crystalline silica, fumed silica, silica gel, and silica aerogel.

Sand surfaces suitable for use in the disclosure include, for example, sand comprised of silica (e.g., quartz), calcium carbonate (e.g., aragonite), and mixtures thereof. The sand can comprise other components, such as minerals (e.g., magnetite, chlorite, glauconite, gypsum, olivine, garnet), metal (e.g., iron), shells, coral, limestone, and rock.

Wood surfaces suitable for the disclosure include, for example, hard wood and soft wood, and materials engineered from wood, wood chips, or fiber (e.g., plywood, oriented strand board, laminated veneer lumber, composites, strand lumber, chipboard, hardboard, medium density fiberboard). Types of wood include alder, birch, elm, maple, willow, walnut, cherry, oak, hickory, poplar, pine, fir, and combinations thereof.

Fiber surfaces suitable for use in the disclosure include, for example, natural fibers (e.g., derived from an animal, vegetable, or mineral) and synthetic fibers (e.g., derived from cellulose, mineral, or polymer). Suitable natural fibers include cotton, hemp, jute, flax, ramie, sisal, bagasse, wood fiber, silkworm silk, spider silk, sinew, catgut, wool, sea silk, wool, mohair, angora, and asbestos. Suitable synthetic fibers include rayon, modal, and Lyocell, metal fiber (e.g., copper, gold, silver, nickel, aluminum, iron), carbon fiber, silicon carbide fiber, bamboo fiber, seacell, nylon, polyester, polyvinyl chloride fiber (e.g., vinyon), polyolefin fiber (e.g., polyethylene, polypropylene), acrylic polyester fiber, aramid (e.g., TWARON™, KEVLAR™, or NOMEX™) and spandex.

Natural polymer surfaces suitable for use in the disclosure include, for example, a polysaccharide (e.g., cotton, cellulose), shellac, amber, wool, silk, natural rubber, and a biopolymer (e.g., a protein, an extracellular matrix component, collagen).

Synthetic polymer surfaces suitable for use in the disclosure include, for example, polyvinylpyrrolidone, acrylics, acrylonitrile-butadiene-styrene, polyacrylonitrile, acetals, polyphenylene oxides, polyimides, polystyrene, polypropylene, polyethylene, polytetrafluoroethylene, polyvinylidene fluoride, polyvinyl chloride, polyethylenimine, polyesters, polyethers, polyamide, polyorthoester, polyanhydride, polysulfone, polyether sulfone, polycaprolactone, polyhydroxy-butyrate valerate, polylactones, polyurethanes, polycarbonates, polyethylene terephthalate, as well as copolymers and combinations thereof.

Typical rubber surfaces suitable for use in the disclosure include, for example, silicones, fluorosilicones, nitrile rubbers, silicone rubbers, polyisoprenes, sulfur-cured rubbers, butadiene-acrylonitrile rubbers, isoprene-acrylonitrile rubbers, and the like.

Ceramic surfaces suitable for use in the disclosure include, for example, boron nitrides, silicon nitrides, aluminas, silicas, combinations thereof, and the like.

Stone surfaces suitable for use in the disclosure include, for example, granite, quartz, quartzite, limestone, dolostone, sandstone, marble, soapstone, and serpentine.

For purposes of the present disclosure, animal bodies include, but are not limited to, the order Rodentia (e.g., mice), the order Logomorpha (e.g., rabbits), the order Camivora (e.g., Felines (cats) and Canines (dogs)), the order Artiodactyla (e.g., Bovines (cows) and Swines (pigs)), the order Perssodactyla (e.g., Equines (horses)), the order Primates, Ceboids, or Simioids (e.g., monkeys), the class Aves (e.g., birds), the class of Phylum Arthropoda (e.g., insects), the class of Pisces (e.g., fish), or the order Anthropoids (e.g., humans and apes).

The surface typically is a component of a larger structure. For example, the surface can be part of a medical device, diagnostic equipment, implant, glove, mask, curtain, mattress, sheets, blankets, gauze, dressing, tissue, surgical drape, tubing, surgical instrument, safety gear, fabric, apparel item, floor, handles, wall, sink, shower or tub, toilet, furniture, wall switch, toy, athletic equipment, playground equipment, shopping cart, countertop, appliance, railing, door, air filter, pipe, utensil, dish, cup, container, object display container, food, food display container, food package, food processing equipment, food handling equipment, food transportation equipment, food vending equipment, food storage equipment, food packaging equipment, plant, phone, cell phone, remote control, computer, mouse, keyboard, touch screen, leather, cosmetic, cosmetic making equipment, cosmetics storage equipment, cosmetics packaging equipment, personal care item, personal care item making equipment, personal care storage equipment, personal care packaging equipment, animal care item, animal care item making equipment, veterinary equipment, powder, cream, gel, salve, eye care item, eye care item making equipment, contact lens, glasses, eye care storage equipment, contact lens case, jewelry, jewelry making equipment, jewelry storage equipment, animal housing, farming equipment, animal food handling equipment, animal food storage space, animal food storage equipment, animal food container, air vehicle, land vehicle, air processing equipment, air filter, water vehicle, water storage space, water storage equipment, water processing equipment, water storage container, water filter, hand, hair, foot, leg, arm, torso, head, or animal body part, pharmaceuticals display container, pharmaceuticals package, pharmaceuticals processing equipment, pharmaceuticals handling equipment, pharmaceuticals transportation equipment, pharmaceuticals vending equipment, pharmaceuticals, pharmaceuticals storage equipment, pharmaceuticals packaging equipment.

A “medical device” includes any device having surfaces that contact tissue, blood, or other bodily fluids in the course of their use or operation, which are found on or are subsequently used within a mammal (e.g., a human). Medical devices include, for example, extracorporeal devices for use in surgery, such as blood oxygenators, blood pumps, blood storage bags, blood collection tubes, blood filters including filtration media, dialysis membranes, tubing used to carry blood and the like which contact blood which is then returned to the patient or mammal. Medical devices also include endoprostheses implanted in a mammal (e.g., a human), such as vascular grafts, stents, pacemaker leads, surgical prosthetic conduits, heart valves, and the like, that are implanted in blood vessels or the heart. Medical devices also include devices for temporary intravascular use such as catheters, guide wires, amniocentesis and biopsy needles, cannulae, drainage tubes, shunts, sensors, transducers, probes and the like which are placed into the blood vessels, the heart, organs or tissues for purposes of monitoring or repair or treatment. Medical devices also include prostheses such as artificial joints such as hips or knees as well as artificial hearts. In addition, medical devices include penile implants, condoms, tampons, sanitary napkins, ocular lenses, sling materials, sutures, hemostats used in surgery, antimicrobial materials, surgical mesh, transdermal patches, and wound dressings/bandages.

The “diagnostic equipment” includes any device or tool used to diagnose or monitor a medical condition. Examples include an ultrasound, MRI machine, PET scanner, CT scanner, ventilator, heart-lung machine, ECMO machine, dialysis machine, blood pressure monitor, otoscope, ophthalmoscope, stethoscope, sphygmomanometer, blood pressure cuff, electrocardiograph, thermometer, defibrillator, speculum, sigmoidoscope, and anoscope.

The “surgical instrument” includes any tool or device used for performing surgery or an operation. Examples include a scalpel, lancet, trocar, hemostat, grasper, forceps, clamp, retactor, distractor, positioner, tracheotome, dilator, stapler, irrigation needle, injection needle, drill, scope, endoscope, probe, ruler, and caliper.

“Safety gear” includes devices used to protect a person, animal, or object. Examples of “safety gear” are a mask, face shield, visor, goggles, glasses, gloves, shoe covers, foot guard, leg guard, belt, smock, apron, coat, vest, raingear, hat, helmet, chin strap, hairnet, shower cap, hearing protection (ear plugs, ear muffins, hearing bands), respirator, gas mask, supplied air hood, collar, leash, and first aid kit.

“Fabric” includes any type of suitable fabric, such as bedding, curtains, towels, table coverings, protective sheeting, and dish cloths.

An “apparel item” includes an item of clothing, footwear, or other item someone would wear on his/her person. Examples include a uniform, coat, shirt, pants, waders, scrubs, socks, shoe or boot liner, an insole, gloves, hats, shoes, boots, and sandals.

The surface can be part of a building structure or an item that can be found in a building structure, such as a floor, wall, an appliance (e.g., a refrigerator, oven, stove, dishwasher, washing machine, clothes dryer, furnace, water heater, air conditioner, heater), sink, shower or tub, toilet, furniture (e.g., mattress, couch, sofa, chair, table, shelf, mantle, bed, dresser), countertop, railing, air filter, air processing equipment, water processing equipment, water filter, pipe, or door.

The surface can also be a toy or athletic equipment, including exercise equipment, playground equipment, or a pool.

The surface can be a utensil (e.g., knife, fork, spoon, ladle, spatula, whisk, etc.), a dish (e.g., a food storage container, a food serving piece, etc.), a food package (e.g., a bag, a box, foil, plastic wrap), or other item that comes in contact with food (e.g., a cutting board, food display container, food processing equipment, food handling equipment, food transportation equipment, food vending equipment, animal food handling equipment, animal food storage space, food storage equipment, animal food container, animal food storage equipment). The surface can be part of food processing equipment, such as food processing tanks, stirrers, conveyor belts, knives, grinders, packaging machines, labeling machines, etc.

The “food” is any food in which it would be desirable to provide with a microbicidal polymer. In such embodiments, the microbicidal polymer and the composition thereof should be nontoxic for human and animal consumption. The “food” can be, e.g., any fruit, vegetable, or meat.

The “plant” is any suitable plant, including an angiosperm (a flowering plant), gymnosperm (a seed-producing plant), a conifer, fern, and moss. Suitable angiosperms are from the amborella (e.g., Amborella trichopoda Baill), nymphaeales (e.g., water lily), austrobaileyales (e.g., Illicium verum), chloranthales (e.g., from the genus ascarina, chloranthus, hedyosmum, or sarcandra), magnoliids (e.g., magnolia, bay laurel, black pepper), monocots (e.g., grasses, orchids, palms), ceratophyllum (e.g., aquatic plants), or eudicots (e.g., sunflower, petunia, apple) groups. Suitable gymnosperms are from the subclass cycadidae, ginkgoidae, gnetidae, or pinidae.

The surface can be part of an electronic device, such as a phone, cell phone, remote control, computer, mouse, keyboard, and touch screen.

The surface can further be part of a cosmetic (e.g., eye shadow, eyeliner, primer, foundation, lipstick, lip gloss, blush), a personal care item (e.g., lip balm, body soap, facial soap, lotion, cologne, perfume, antiperspirant, deodorant, facial tissue, cotton swabs, cotton pads, mouthwash, toothpaste, nail polish, shampoo, conditioner, hairspray, talcum powder, shaving cream, contact lens, contact lens case, glasses), or jewelry (e.g., necklace, ring, earring, bracelet, watch).

The “animal care item” and “veterinary equipment” can be any product used in a setting that includes animals, such as a house, boarding house, or veterinary hospital. Of course, veterinary equipment can be used at a location outside of a hospital setting. Animals are any animals that are typically considered pets, non-pets, boarded, or treated by a veterinarian. Examples of suitable animals include a dog, cat, reptile, bird, rabbit, ferret, guinea pig, hamster, rat, mouse, fish, turtle, horse, goat, cattle, and pig. Suitable pet care items include the personal care items described herein, toys, bed, crate, kennel, carrier, bowl, dish, leash, collar, litterbox, and grooming items (e.g., clippers, scissors, a brush, comb, dematting tool, and deshedding tool). Suitable veterinary equipment includes any of the medical devices and surgical instruments described herein and other equipment, such as a table, tub, stretcher, sink, scale, cage, carrier, and leash.

The “animal housing” can be any suitable housing, such as a coop, stable, shelter, grab bag shelter, hutch, barn, shed, pen, nestbox, feeder, stanchion, cage, carrier, or bed.

The “farming equipment” is any device used in an agricultural setting, including a farm or ranch, particularly a farm or ranch that houses animals, processes animals, or both. Animal livestock that can be housed or processed as described herein and include, e.g., horses, cattle, bison, and small animals such as poultry (e.g., chickens, quails, turkeys, geese, ducks, pigeons, doves, pheasants, swan, ostrich, guineafowl, Indian peafowl, emu), pigs, sheep, goats, alpacas, llamas, deer, donkeys, rabbits, and fish. Examples of farming equipment include as a wagon, trailer, cart, barn, shed, fencing, sprinkler, shovel, scraper, halter, rope, restraining equipment, feeder, waterer, trough, water filter, water processing equipment, stock tank, fountain, bucket, pail, hay rack, scale, poultry flooring, egg handling equipment, a barn curtain, tractor, seeder, planter, plow, rotator, tiller, spreader, sprayer, agitator, sorter, baler, harvester, cotton picker, thresher, mower, backhoe loader, squeeze chute, hydraulic chute, head chute, head gate, crowding tub, corral tub, alley, calving pen, calf table, and milking machine.

The surface can be part of a vehicle, such as an air vehicle, land vehicle, or water vehicle. Suitable vehicles include a car, van, truck, bus, ambulance, recreational vehicle, camper, motorcycle, scooter, bicycle, wheelchair, train, streetcar, ship, boat, canoe, submarine, an unmanned underwater vehicle (UUV), a personal water craft, airplane, jet, helicopter, unmanned autonomous vehicle (UAV), and hot air balloon.

If desired, the surface to which the polymer has been applied can be regenerated by removing the polymer coating. Thus, the polymer coating described herein can be considered temporary. To renew the surface, any of the methods described herein can further comprise removing the polymer from the surface by washing the surface with a second solvent or simply wiping away the polymer coating. The washing step further serves to remove any microbe corpses and/or other potentially harmful biological debris from the surface.

The second solvent suitable for washing can be the same as the solvent used for polymer application to a surface as described above. In certain aspects, the second solvent comprises water, organic solvents, such as alcohols, or a mixture thereof. In some embodiments, the second solvent can comprise a lower alcohol, such as methanol, ethanol, n-propanol, isopropanol, or a combination thereof. In some embodiments, the second solvent is the same as the solvent present in the original polymer composition used to apply the polymer to the surface. In some embodiments, the polymer is soluble in the second solvent.

The following examples further illustrate the disclosure but, of course, should not be construed as in any way limiting its scope.

### EXAMPLES

### Example 1

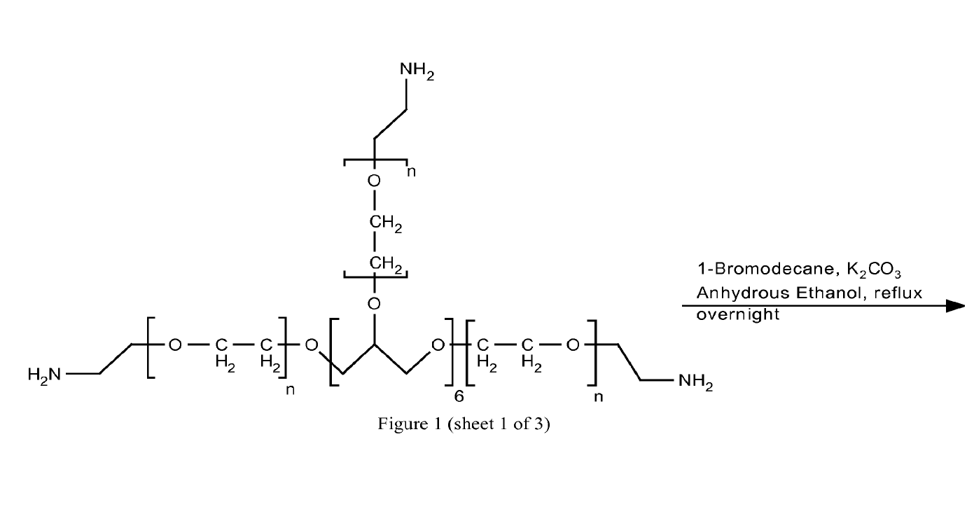

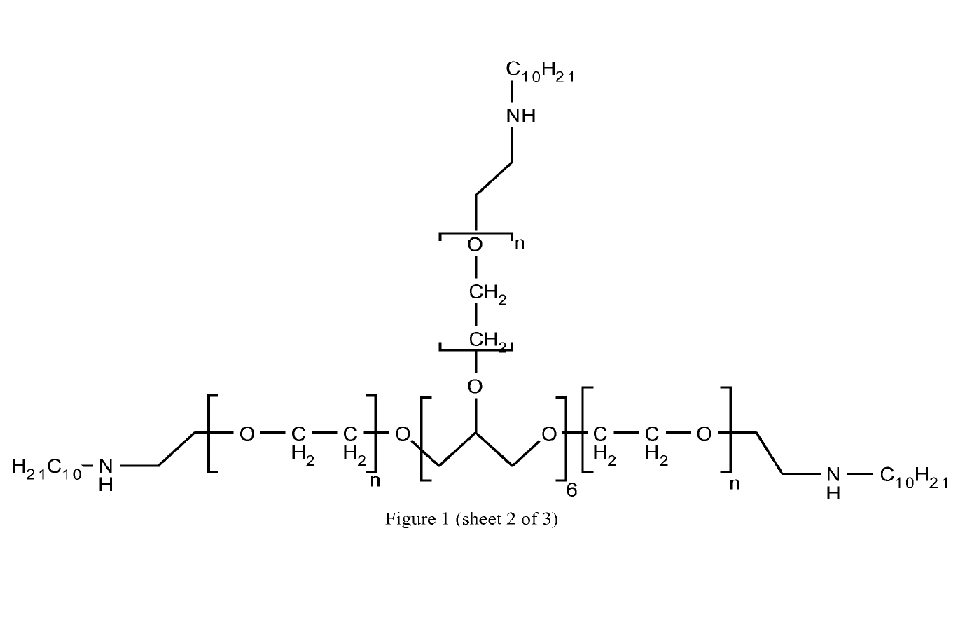

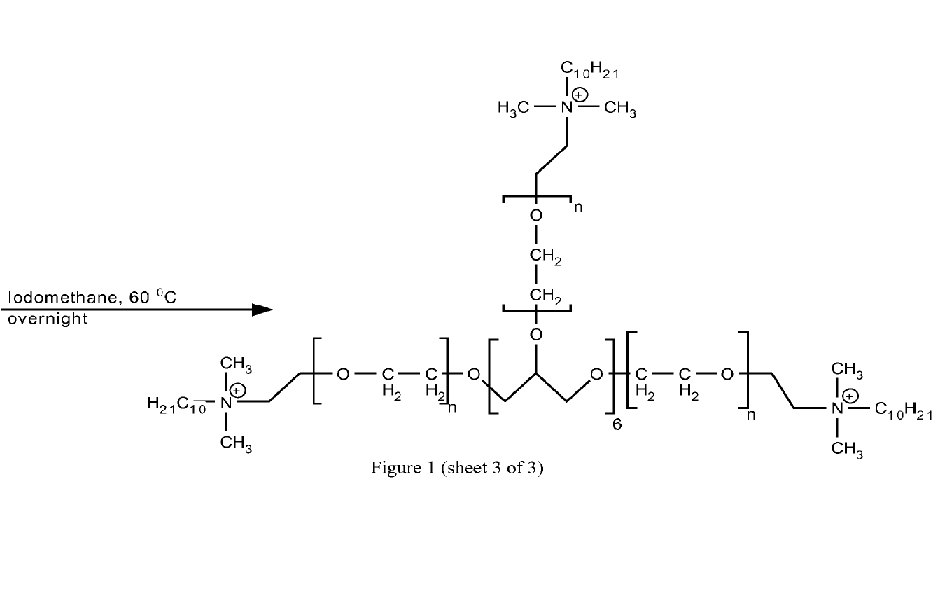

This example demonstrates a synthesis of N,N-decyl,methyl-PEG in an aspect of the disclosure. The synthesis is set forth in FIG. 1. The starting PEG material (molecular weight: 10,000 g/mol (“10 k”)) was thoroughly dried in order to completely remove water. The drying process was performed using a lyophilizer over a period of 4-5 days or a vacuum line at high temperature (about 65° C.) for about 3 hours. Dry PEG (0.733 g, 0.0733 mmol, 0.586 mmol eq. of NH₂) was mixed with potassium carbonate (0.8104 g, 10 eq., 5.86 mmol) in a 100 ml round bottom flask, along with 50 ml of anhydrous ethanol and 1-bromodecane (1.216 ml, 10 eq., 5.86 mmol).

The resulting mixture was magnetically stirred overnight at refluxing temperature. The mixture was cooled to room temperature, and the resulting suspension was filtered through a frit funnel. Iodomethane (4.159 g, 50 eq., 29.3 mmol), as 14.65 mL of a 2 M solution in t-butyl methyl ether, was added to the filtrate, and the resulting mixture was further stirred at 60° C. overnight. The reaction mixture was then cooled a second time back to room temperature and concentrated on the rotary evaporator at 80° C. before the addition of hexanes. The hexanes counter solvent caused the precipitation of the desired product. The resulting suspension was filtered through a frit funnel, and the product was washed with hexanes, and sonicated in hexanes. The final product was the polymer having the polycation N,N-decyl,methyl-PEG(10 k, 8-arm).

### Example 2

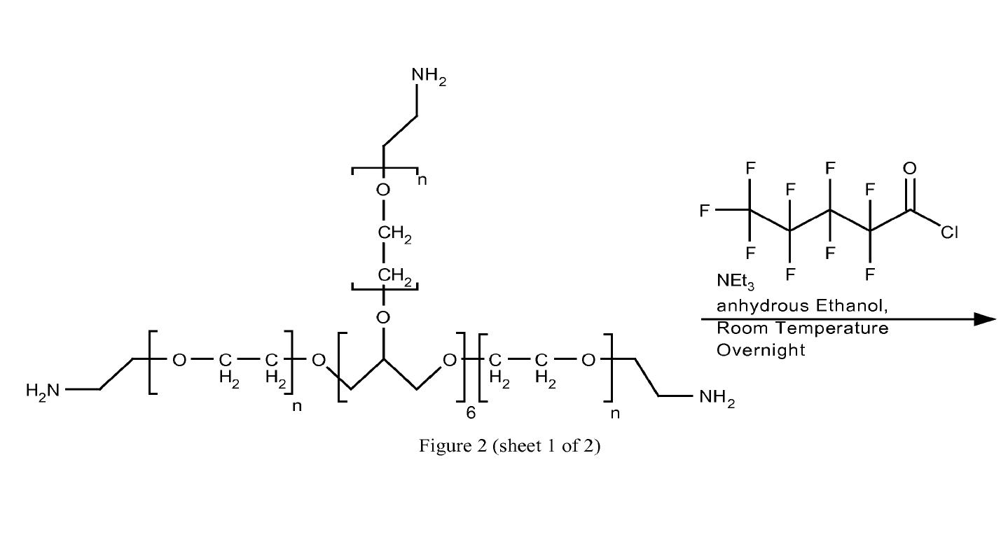

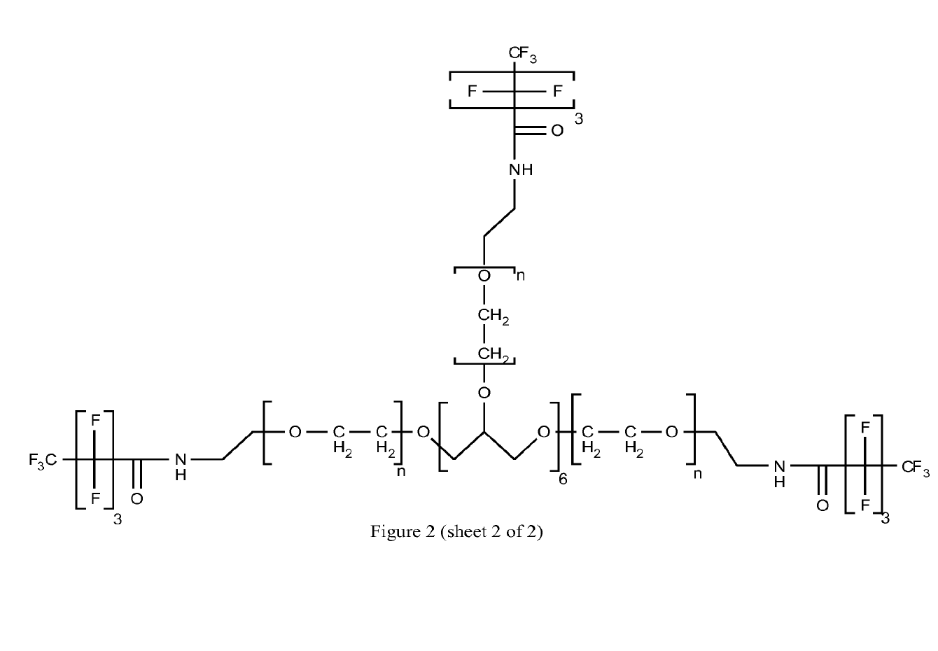

This example demonstrates a synthesis of N-nonafluorobutylamide-PEG in an aspect of the disclosure. The synthesis is set forth in FIG. 2. The starting PEG material (molecular weight: 10,000 g/mol (“10 k”)) is thoroughly dried in order to completely remove water. Dry PEG (0.2 g, 0.02 mmol, 0.16 mmol eq. of NH₂) is mixed with triethylamine (33.4 μL, 1.5 NH₂— eq., 0.24 mmol) in a 100 ml round bottom flask, along with 50 ml of anhydrous ethanol, and nonafluoropentanoyl chloride (45.2 mg, 1 NH₂— eq., 0.16 mmol). The final mixture is magnetically stirred at room temperature overnight.

On the following day, the mixture is evaporated on the rotary evaporator at 80° C. in order to remove all volatile materials. The product is then dissolved back into 50 ml ethanol before adding hexanes. The hexanes counter solvent causes the precipitation of the desired product. The suspension is filtered through a frit funnel, and the product is washed with hexanes and then sonicated in hexanes. The final product is N-nonafluorobutylamide-PEG(10 k, 8-arm).

### Example 3

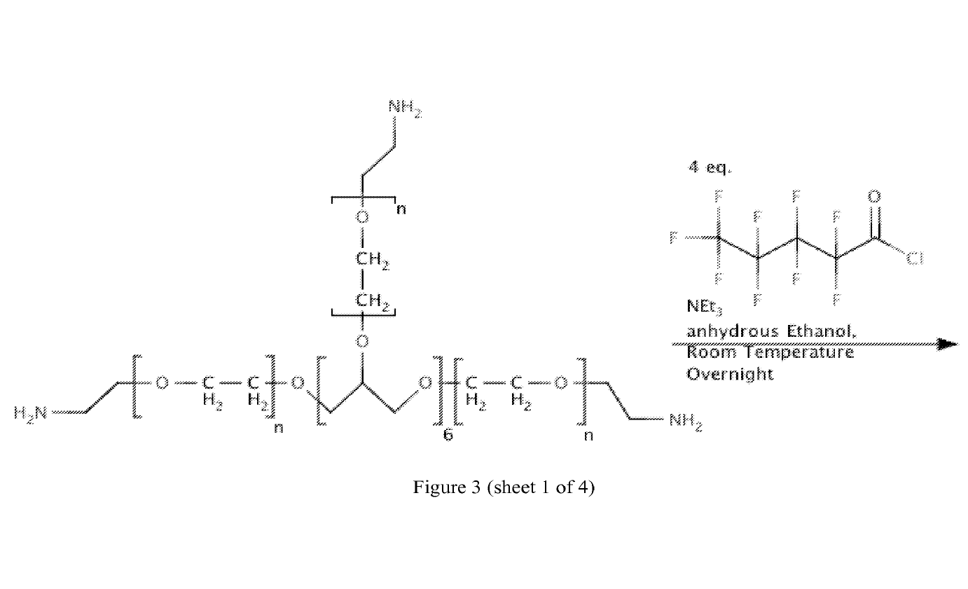

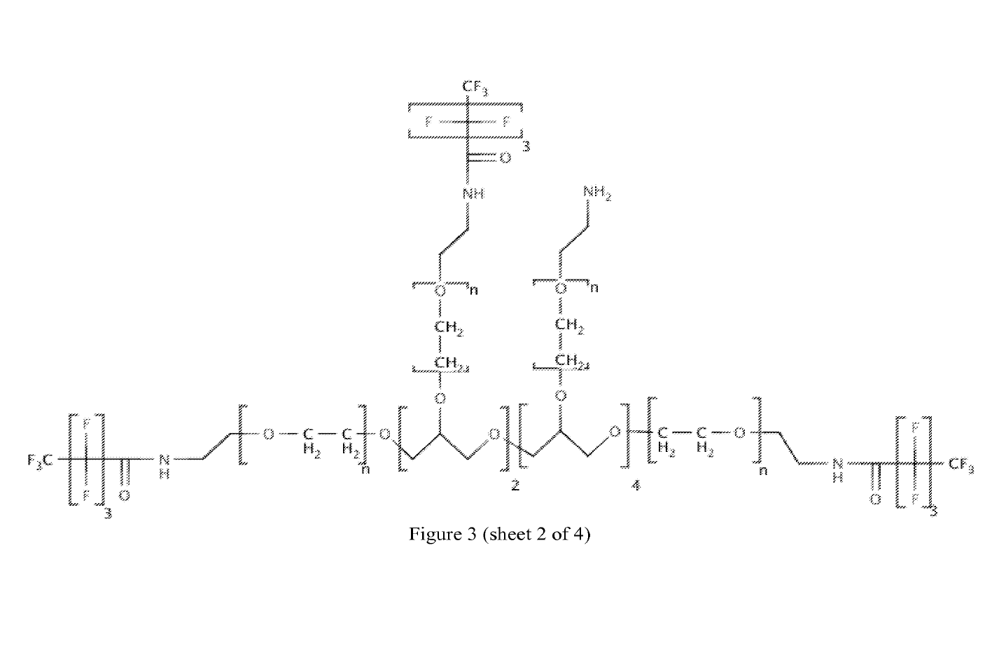

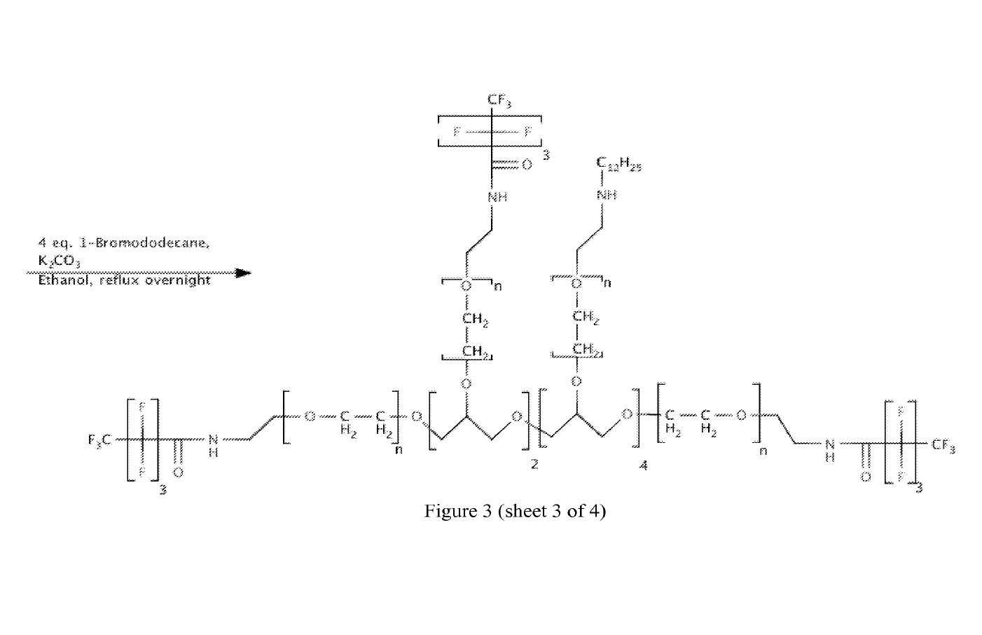

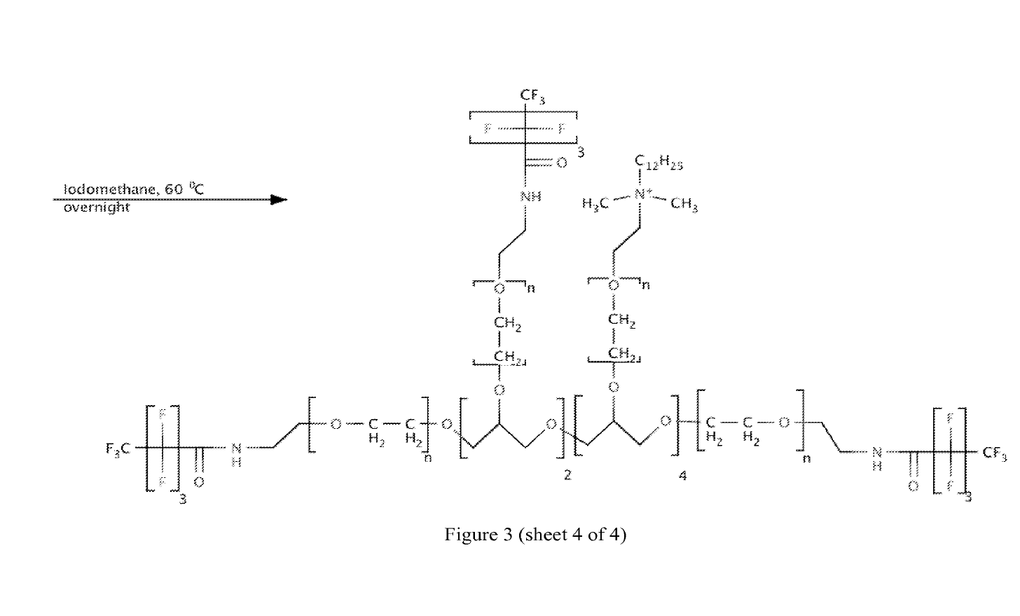

This example demonstrates a synthesis of polycation (N,N-dodecyl,methyl)(N-nonafluorobutylamide)-PEG in an aspect of the disclosure. The synthesis is set forth in FIG. 3. The starting PEG material (molecular weight: 10,000 g/mol (“10 k”)) is thoroughly dried in order to completely remove water. Dry PEG (0.2 g, 0.02 mmol, 0.16 mmol eq. of NH₂) is mixed with triethylamine (16.7 μL, 0.75 NH₂— eq., 0.12 mmol) in a 100 ml round bottom flask, along with 50 ml of anhydrous ethanol, and nonafluoropentanoyl chloride (22.6 mg, 0.5 NH₂— eq., 0.08 mmol). The final mixture is magnetically stirred at room temperature overnight.

On the following day, the mixture is evaporated on the rotary evaporator at 80° C. in order to remove all volatile materials. The product is then dissolved back into 50 ml ethanol before adding hexanes. The hexanes counter solvent causes the precipitation of the desired product. The suspension is filtered under vacuum through a frit funnel, and the product is washed with hexanes.

The intermediate semi-functionalized N-nonafluorobutylamide-PEG (0.02 mmol, 0.08 mmol eq. of NH₂) is mixed with potassium carbonate (0.110 g, 10 eq., 0.8 mmol) in a 100 ml round bottom flask, along with 50 ml of anhydrous ethanol and 1-bromododecane (0.192 ml, 10 eq., 0.8 mmol).

The final mixture is magnetically stirred overnight at refluxing temperature. The mixture is then cooled to room temperature, and the resulting suspension is filtered through a frit funnel. Iodomethane (0.568 g, 50 eq., 4 mmol) as 2 mL of a 2 M solution in t-butyl methyl ether is added to the filtrate, and the resulting mixture is further stirred at 60° C. overnight. The reaction mixture is then cooled a second time back to room temperature before the addition of hexanes. The hexanes counter solvent causes the precipitation of the desired product. The suspension is filtered through a frit funnel, and the product is washed with hexanes and then sonicated in hexanes. The final product is the polymer having polycation (N,N-dodecyl,methyl)₄(N-nonafluorobutylamide)₄-PEG(10 k, 8-arm).

### Example 4

This example demonstrates the microbicidal effect of a polymer having the polycation N,N-decyl,methyl-PEG coated on a surface in an aspect of the disclosure.

Preparation of the Escherichia coli (E. Coli) Inoculum

Lysogeny broth (LB) (1 mL) was added to a 15 ml cell culture tube. Seven (7) μl of 40% glycerol stock of the ONE SHOT™ TOP10 E. Coli cells (Life Technologies, Grand Island, N.Y.) (from the −20° C. freezer) was inoculated into the 1 ml LB broth. The inoculate was incubated at 37° C. for 3 hours with constant shaking (ca. 250 rpm). The inoculate was then centrifuged at 2500 rpm speed for 4 min. The LB broth was separated, and the E. coli cells were washed twice with 1 ml of sterile phosphate buffered saline (PBS) 1× (pH 7.4) each time and then re-suspended in 1 mL of the same buffer. Using optical density measurements, the final concentration was estimated to be 10⁷-10⁸ cells/ml.

### Preparing LB Agar Medium (1 L):

In a 1 L French style bottle equipped with a magnetic stirrer was added 800 ml dH₂O (deionized MILLI-Q™ water), in which 10 g tryptone (BD 211705, pancreatic digest of casein), 5 g yeast extract (BD 212750, extract from autolysed yeast cells), 5 g NaCl (Sigma Aldrich, St. Louis, Mo.) was dissolved. The pH was checked and adjusted to 7.0 with a concentrated NaOH (1-5 M), if necessary. There was no need for pH adjustment. dH₂O was added to provide a final volume of 1000 ml. Agar powder (15 g) (final concentration=1.5%) (BD 214530, DIFCO™ Agar Granulated) was added. The mixture was stirred to provide the complete dissolution (or at least suspension) of agar into solution. The mixture was sterilized by autoclaving at 15 psi and 121-124° C. for 15-25 minutes. The resulting mixture was cooled to 45° C. in a water bath.

Coating Glass Slides with N,N-decyl,methyl-PEG (“RAN-001-129”) Polymer:

The first set of two slides were untreated (slides RAN-001-141-1) as a control. Two glass slides (ca. 10 cm×2 cm) were coated with a 70% isopropanol solution in water (slides RAN-001-141-2) as a control. Two glass slides (ca. 10 cm×2 cm) were coated with a solution of 100 mg of RAN-001-129 in 1 L of 70% isopropanol (slides RAN-001-141-3). Coating was achieved by spreading 300 μL of the solution with a plastic pipette tip, allowing the solution to sit for 5 min, then draining the solution, followed by air drying for 30 min inside a fume hood.

### Applying E. Coli Inoculum to Treated and Untreated Glass Slides:

About 100 μL of the E. Coli solution in PBS was spread over each slide with a plastic pipette tip. The solution was allowed to dry for 5 min in air. The E. Coli solution was covered with an LB agar layer by pouring the 45° C. warm solution on top of the slide and the rest of the plate. The slide was allowed to dry inside a biosafety cabinet for 30 min, and then incubated at 37° C. overnight.

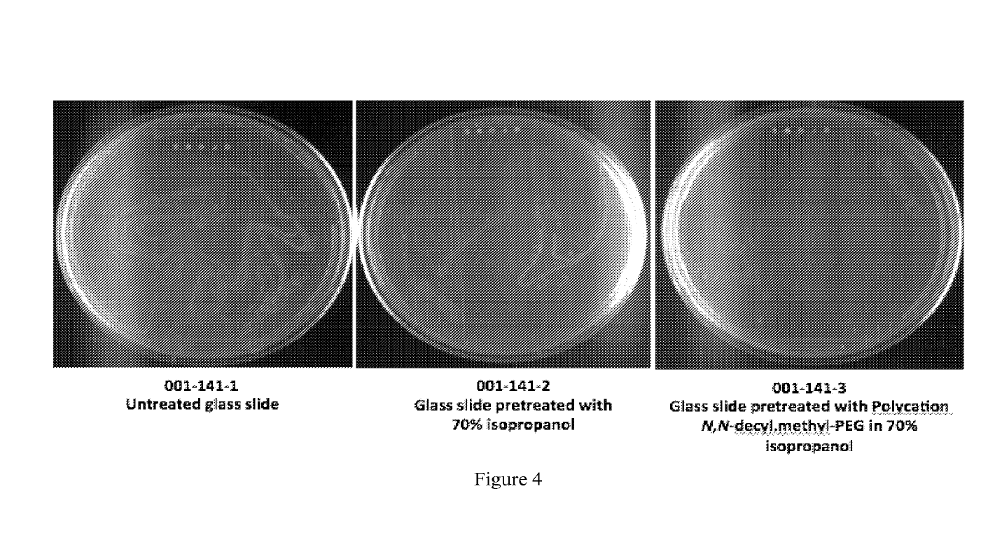

On the following day, there was limited-to-no bacterial growth on the glass surface on which the polymer RAN-001-129 was applied (slides RAN-001-141-3). See FIG. 4. As seen in FIG. 4(c), the presence of the polymer reduced the growth of E. Coli relative to the controls in FIGS. 4(a) and 4(b). In addition, the microbicidal influence of the polymer extended beyond the treated glass surface. Since the polymer is soluble in aqueous solutions, such as agar, the polymer may have desorbed off the surface.

### Example 5

This example further demonstrates the microbicidal effect of the polymer having polycation N,N-decyl,methyl-PEG coated on a surface in an aspect of the disclosure.

Preparation of Escherichia coli (E. Coli) Inoculum

An E. Coli inoculum was prepared as described in Example 4. Using optical density measurements, the final concentration was estimated to be 10⁹ cells/ml.

### Preparing LB Agar Medium (1 L):

An LB agar medium was prepared as described in Example 4.

Coating Glass Slides with N,N-decyl,methyl-PEG (“RAN-001-129”) Polymer:

The first set of two slides were untreated (slides RAN-001-144-1) as a control. Two glass slides (ca. 10 cm×2 cm) were coated with a 70% isopropanol solution in water (slides RAN-001-144-2) as a control. Two glass slides (ca. 10 cm×2 cm) were coated with a solution of 1 mg of RAN-001-129 (from Example 1) in 1 mL of 70% isopropanol (slides RAN-001-144-3). Coating was achieved by spreading 300 μL of the solution using the tip of a plastic pipette, and allowing the solution to dry over 1 h.

### Applying E. Coli Inoculum to Treated and Untreated Glass Slides:

About 50 μL of the E. Coli solution in PBS was spread over each slide, using the tip of a plastic pipette. The solution was allowed to dry for 20 min in air. The E. Coli solution was covered with an LB agar layer by pouring the 45° C. warm solution on top of the slide and the rest of the plate. The slide was allowed to dry inside a biosafety cabinet for 45 min, and then incubated at 37° C. overnight.

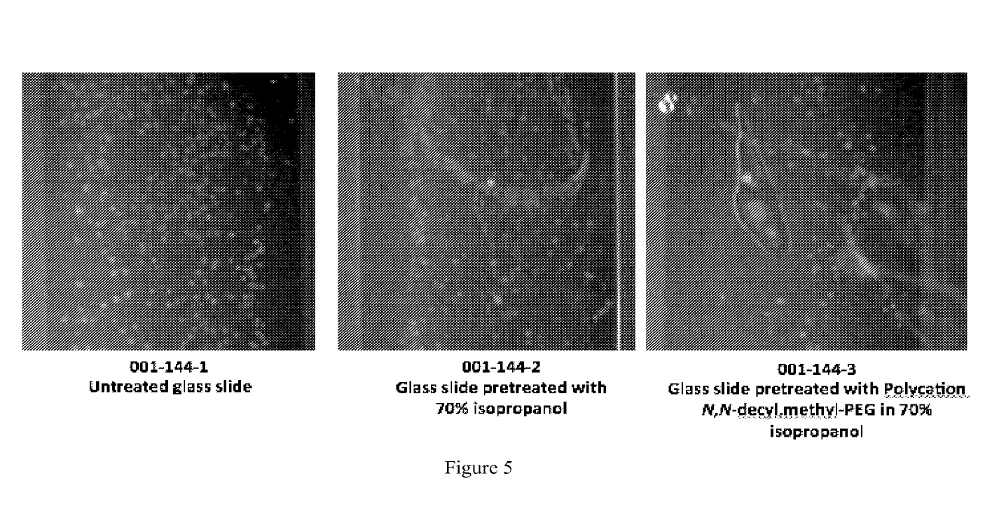

On the following day, bacterial growth was reduced on the glass surface on which the polymer RAN-001-129 was applied (slides RAN-001-144-3). See FIG. 5. As seen in FIG. 5(c), the presence of the polymer reduced the growth of E. Coli relative to the controls in FIGS. 5(a) and 5(b).

### Example 6

This example demonstrates the microbicidal effect of various concentrations of polycation N,N-decyl,methyl-PEG polymer coated on a surface in an aspect of the disclosure.

Preparing Escherichia coli (E. Coli) Inoculum

An E. Coli inoculum was prepared as described in Example 4. Using optical density measurements, the final concentration was estimated to be 10⁸ cells/ml.

### Preparing LB Agar Medium (1 L):

An LB agar medium was prepared as described in Example 4.

Coating Glass Slides with Polycation N,N-decyl,methyl-PEG (“RAN-001-156”) Polymer:

The first set of two slides were untreated (159-1A and 159-1B) as a control. Two glass slides (ca. 2.5 cm×2.5 cm) were coated with a 70% isopropanol solution in water (159-2A and 159-2B) as a control. Glass slides (ca. 2.5 cm×2.5 cm) were coated with a solution of RAN-001-156 (from Example 1) in 70% isopropanol using the concentrations set forth in Table 1. Coating was achieved by spreading 300 μL of the solution using the tip of a plastic pipette, and allowing the solution to dry over 1 h.

### Applying E. Coli Inoculum to Treated and Untreated Glass Slides:

About 20 μL of the E. Coli solution (about 10⁸ cells/mL) in PBS was spread over each slide, using the tip of a plastic pipette. The solution was allowed to dry for 20 min in air. The E. Coli solution was covered with 7.5 mL of LB agar in a 60 mL plate. The slide was incubated at 37° C. overnight.

On the following day, bacterial growth was reduced on the glass surface on each slide in which the polymer RAN-001-129 was applied (slides 159-3A/B through 159-6A/B). See Table 1.

TABLE 1

| Name | Composition | Result |
|---|---|---|
| 159-1A and | No coating | Bacterial growth |
| 159-1B | | |
| 159-2A and | 70% isopropanol | Bacterial growth |
| 159-2B | | |
| 159-3A and | 1 mg of RAN-001-159 in 10 mL | No bacterial growth |
| 159-3B | 70% isopropanol | |
| 159-4A and | 5 mg of RAN-001-159 in 10 mL | No bacterial growth |
| 159-4B | 70% isopropanol | |
| 159-5A and | 10 mg of RAN-001-159 in 10 mL | No bacterial growth |
| 159-5B | 70% isopropanol | |
| 159-6A and | 50 mg of RAN-001-159 in 10 mL | No bacterial growth |
| 159-6B | 70% isopropanol | |

Thus, a microbidical effect was observed with concentrations as low as 1 mg of polymer in 10 mL of 70% isopropanol.

### Example 7

This example demonstrates the microbicidal effect of polymer having polycation N,N-decyl,methyl-PEG coated on a surface with varying contact time of the bacterial solution in an aspect of the disclosure.

Preparation of Escherichia coli (E. Coli) Inoculum

An E. Coli inoculum was prepared using the method described in Example 4. Using optical density measurements, the final concentration was estimated to be 10⁸ cells/ml.

### Preparation of LB Agar Medium (1 L):

An LB agar medium was prepared using the method described in Example 4.

Coating Glass Slides with Polycation N,N-decyl,methyl-PEG (“RAN-001-156”) Polymer:

Glass slides (ca. 2.5 cm×2.5 cm) were coated with a solution of 10 mg RAN-001-156 (from Example 1) in 70% isopropanol. Coating was achieved by spreading 300 μL of the solution using the tip of a plastic pipette, and allowing the solution to dry over 1 h.

### Applying E. Coli Inoculum to Treated and Untreated Glass Slides:

About 20 μL of the E. Coli solution (about 10⁸ cells/mL) in PBS was spread over each slide, using the tip of a plastic pipette. The solution was allowed to dry for the times set forth in Table 2. The E. Coli solution was covered with 7.5 mL of LB agar in a 60 mL plate. The slide was incubated at 37° C. overnight.

On the following day, bacterial growth was reduced on the glass surface regardless of the bacteria contact time. See Table 2.

TABLE 2

| | Bacteria Contact | |
|---|---|---|
| Name | Time (min) | Result |
| 159-5A and | 20 | No bacterial growth |
| 159-5B | | |
| 159-5C and | 10 | No bacterial growth |
| 159-5D | | |
| 159-5E and | 45 | No bacterial growth |
| 159-5F | | |

Thus, a microbidical effect was observed with bacterial contact time as low as 10 min.

### Example 8

This example demonstrates the removal of a polycation N,N-decyl,methyl-PEG polymer coating on a surface in an aspect of the disclosure.

Preparation of Escherichia coli (E. Coli) Inoculum

An E. Coli inoculum was prepared using the method described in Example 4. Using optical density measurements, the final concentration was estimated to be 10⁸ cells/ml.

### Preparation of LB Agar Medium (1 L):

An LB agar medium was prepared using the method described in Example 4.

Coating Glass Slides with Polycation N,N-decyl,methyl-PEG (“RAN-001-156”) Polymer:

Glass slides (ca. 2.5 cm×2.5 cm) were coated with a solution of 10 mg RAN-001-156 (from Example 1) in 70% isopropanol. Coating was achieved by spreading 300 μL of the solution using the tip of a plastic pipette, and allowing the solution to dry over 1 h. For slides in which the polymer coat was to be removed, a tissue soaked with water or 70% isopropanol was used to wipe the glass surface. See Table 3 for each of the slide conditions.

### Applying E. Coli Inoculum to Treated and Untreated Glass Slides:

About 20 μL of the E. Coli solution (about 10⁸ cells/mL) in PBS was spread over each slide, using the tip of a plastic pipette. The solution was allowed to dry for 20 min. The E. Coli solution was covered with 7.5 mL of LB agar in a 60 mL plate. The slide was dried inside a biosafety cabinet for 45 min and incubated at 37° C. overnight.

On the following day, it was observed that wiping a polymer-coated surface with a water- or 70%-isopropanol-soaked cloth regenerated the non-microbicidal surface. See Table 3.

TABLE 3

| Name | Conditions | Result |
|---|---|---|
| 159-5A and | 1 coat of polymer and 20 min | No bacterial growth |
| 159-5B | bacteria contact time | |
| 159-5G and | Wipe with water before | Bacterial growth |
| 159-5H | bacteria application (20 min | |
| | bacteria contact time) | |
| 159-5I and | Wipe with 70% isopropanol | Bacterial growth |
| 159-5J | before bacteria application | |
| | (20 min bacteria contact time) | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Some embodiments of this disclosure are described herein, including the best mode known to the inventors for carrying out the disclosure. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A polymer comprising an alkylene oxide backbone to which are attached one or more alkyl or alkylene oxide primary branches, wherein at least one of the alkyl or alkyene oxide primary branches is functionalized with a quaternary ammonium group or a fluorinated group, or at least two of the alkyl or alkylene oxide primary branches are functionalized with a quaternary ammonium group and a fluorinated group, wherein the alkyl or alkylene oxide primary branches optionally contain one or more alkyl or alkylene oxide secondary branches that are functionalized with a quaternary ammonium group or a fluorinated group, wherein said polymer is associated with an anion to maintain electro-neutrality when a quaternary ammonium group is present.

2. The polymer of claim 1, wherein the alkylene oxide backbone comprises a propylene oxide backbone, an ethylene oxide backbone, or a backbone comprising both propylene oxide and ethylene oxide units.

3. The polymer of claim 1, wherein the primary branch comprises at least one ethylene oxide unit or at least one propylene oxide unit.

4. The polymer of claim 1, wherein the quaternary ammonium or fluorinated group is at a terminus of at least one of the alkyl or alkylene oxide primary branches.

5. The polymer of claim 1, wherein the alkylene oxide backbone is linked to at least 2 to 20 alkyl or alkylene oxide primary branches.

6. (canceled)

7. The polymer of claim 2, wherein the propylene oxide backbone has a formula: 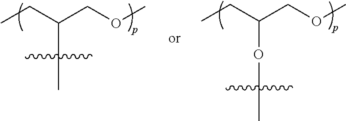
wherein p is 1 to 60.

8. The polymer of claim 1, wherein the alkyl or alkylene oxide primary branch, or the alkyl or alkylene oxide secondary branch, that is functionalized is of the formula
—(CH₂)ₙ—X,—(CH₂CH₂O)ₙ—X, or —(CH₂CH₂CH₂O)ₙ—X,
wherein
X is —(CH₂)ₘ—Y, when Y is a quaternary ammonium group or
X is —(CH₂)ₘ—NHC(O)—Y, when Y is a fluorinated group,
m is 0 to 10, and
n is 1 to 2,500.

9. The polymer of claim 1, wherein at least one of the alkyl or alkylene oxide primary branches is functionalized with a quaternary ammonium group.

10. The polymer of claim 1, wherein the quaternary ammonium group has the formula —N⁺R¹R²R³, wherein R¹, R², and R³ are independently selected from alkyl, alkenyl, cycloalkyl, and aryl.

11-14. (canceled)

15. The polymer of claim 1, wherein at least one of the alkyl or alkylene oxide primary branches is functionalized with a fluorinated group.

16-19. (canceled)

20. The polymer of claim 1, wherein at least two of the alkyl and/or alkylene oxide primary branches are functionalized with a quaternary ammonium group and a fluorinated group.

21. The polymer of claim 1, which is selected from 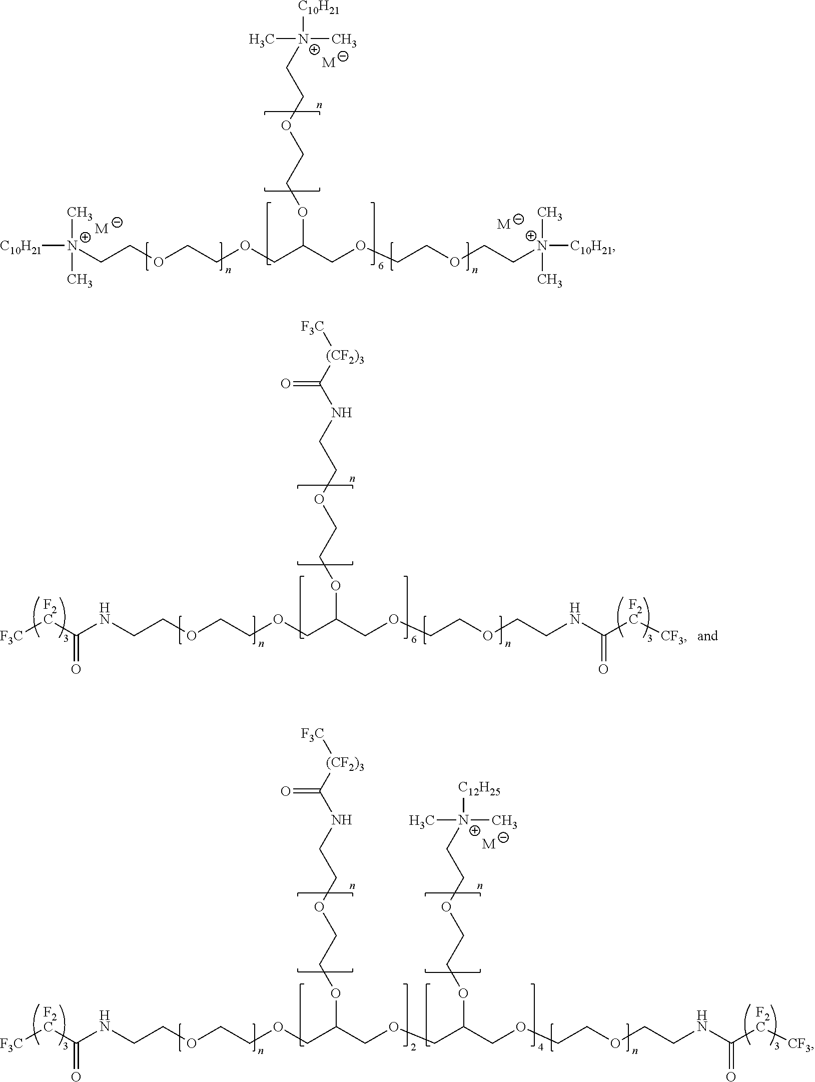
wherein M⁻ is an anion, and n is 2 to 2500.

22. A composition comprising:
(i) a polymer of claim 1; and
(ii) a carrier.

23-26. (canceled)

27. A method of coating a surface, disinfecting a surface, or protecting a surface against growth of a microorganism comprising applying to the surface the composition of claim 22.

28-35. (canceled)

36. The method of claim 27, further comprising removing the polymer from the surface by washing the surface with a second solvent.

37-41. (canceled)

42. A coated surface comprising a surface and a composition of claim 22 coated on the surface.

43-48. (canceled)

49. A method of adjusting the solvation of the composition of claim 22 comprising selecting an appropriate alkylene oxide and alkyl groups in the backbone and branches and their number of repeat units and density of branches, and selecting a quaternary ammonium group or groups and/or a fluorinated group or groups to provide a polymer that is soluble in water, organic solvent, or a mixture thereof.

50. A method of adjusting the hydrophobicity, hydrophilicity, and/or fluorophilicity of the composition of claim 22 comprising selecting an appropriate alkylene oxide and alkyl groups in the backbone and branches and their number of repeat units and density of branches, and selecting a quaternary ammonium group or groups and/or a fluorinated group or groups to provide a polymer that is hydrophobic, hydrophilic, and/or fluorophilic.

51. A method of adjusting the hydrophobicity, hydrophilicity, fluorophilicity, and/or microbicidal performance of the coated surface of claim 42 comprising selecting an appropriate concentration of polymer on the surface by varying the polymer concentration in the carrier and by varying the number and methods of polymer coating.
